(19) United States
(12) Patent Application Publication
(10) Pub. No.: US 2024/0200149 A1
(43) Pub. Date: Jun. 20, 2024

(54) METHOD FOR ANALYZING PROBABILITY OF SUFFERING FROM CANCER IN SUBJECT

(71) Applicant: KABUSHIKI KAISHA TOSHIBA, Tokyo (JP)

(72) Inventors: Tomomi ANDO, Ota (JP); Mitsuko ISHIHARA, Setagaya (JP); Yoshitake SANO, Adachi (JP); Takuya MIYAGAWA, Yokohama (JP); Koji HASHIMOTO, Atsugi (JP)

(73) Assignee: KABUSHIKI KAISHA TOSHIBA, Tokyo (JP)

(21) Appl. No.: 18/591,077

(22) Filed: Feb. 29, 2024

Related U.S. Application Data
(63) Continuation of application No. PCT/JP23/31406, filed on Aug. 30, 2023.

(30) Foreign Application Priority Data
Sep. 20, 2022 (JP) 2022-149024

(51) Int. Cl. C12Q 1/6886; C12Q 1/6874

(52) U.S. Cl. CPC C12Q 1/6886; C12Q 1/6874; C12Q 2600/16; C12Q 2600/178

(57) ABSTRACT

According to one embodiment, a method for analyzing the probability of suffering from cancer in a subject is provided. The method includes counting the number of types of RNA in a sample derived from the subject with respect to the types of RNAs in which sequence variation based on RNA editing exists in comparison with a reference sequence, and determining the probability of suffering from cancer in the subject by using the number of types of RNAs obtained as an index.

### CROSS-REFERENCE TO RELATED APPLICATIONS

This application is a Continuation Application of PCT Application No. PCT/JP2023/031406, filed Aug. 30, 2023 and based upon and claims the benefit of priority from Japanese Patent Application No. 2022-149024, filed Sep. 20, 2022, the entire contents of all of which are incorporated herein by reference.

### FIELD

Embodiments described herein relate generally to a method for analyzing probability of suffering from cancer in a subject.

### BACKGROUND

There are known systems that discriminate between cancer patients and healthy individuals by analyzing nucleic acids extracted from body fluids that can be easily collected. For example, widely tested systems are discrimination systems which use microRNAs (miRNAs). The miRNAs are single-stranded nucleic acids with about 17 to 25 bases and have been known to have the function of regulating gene expression. It has been reported that the types and expression levels of miRNAs vary from the early stages in various diseases. For example, various miRNA levels are used as cancer markers in cancer patients and are known to increase or decrease compared to those of healthy subjects. These findings suggest that quantitative examination of the miRNAs of interest in samples taken from a subject can be a means of knowing whether or not the subject has cancer.

### BRIEF DESCRIPTION OF THE DRAWINGS

FIG. 1 is a scheme diagram showing the first embodiment.

FIG. 2 is a diagram showing one example of the first embodiment.

FIG. 3 is a scheme diagram showing one example of the second embodiment.

FIG. 4 is a scheme diagram showing another example of the second embodiment.

FIG. 5 is a scheme diagram showing the third embodiment.

FIG. 6 is a scheme diagram showing one example of the fourth embodiment.

FIG. 7 is a diagram showing one example of the fourth embodiment.

FIG. 8 is a diagram showing one example of the fourth embodiment.

FIG. 9 is a scheme diagram showing the fifth embodiment.

FIG. 10 is a diagram showing one example of the fifth embodiment.

FIG. 11 is a diagram showing a further example of the fifth embodiment.

FIG. 12 is a diagram showing a further example of the fifth embodiment.

FIG. 13 is a diagram showing results of Example 1.

FIG. 14 is a diagram showing results of Example 2.

FIG. 15 is a diagram showing results of Example 3.

### DETAILED DESCRIPTION

In general, according to one embodiment, according to one embodiment, a method for analyzing probability of suffering from cancer in a subject, comprises: counting the number of types of RNA in a sample derived from the subject with respect to the types of RNAs in which sequence variation caused by RNA editing exists in comparison with a reference sequence, and determining the probability of suffering from cancer in the subject by using the counted number of types of RNAs as an index.

Embodiments will now be described with reference to the accompanying drawings. In each embodiment, the same symbol is designated to a substantially identical configuration part, and the description thereof may be partially omitted. Note further that the drawings are schematic, and the relationship between the thickness of each part and the plane dimensions, the ratio of the thickness of each part, etc. may differ from those in reality.

### First Embodiment

The analytic methods of the embodiments in the present application have been achieved by the findings that it is possible to discriminate between cancer patients and non-cancer individuals by clarifying how the sequence variation caused by RNA editing is present in the RNA group. This discovery makes it possible to analyze the probability of a subject having cancer qualitatively rather than quantitatively. In this way, it is possible to detect cancer at an early stage more easily and, consequently, less expensively. For example, since there is no need to quantify genes, there is no need to ensure quantitative performance. These analytical methods are breakthrough and revolutionary methods based on a very original discovery.

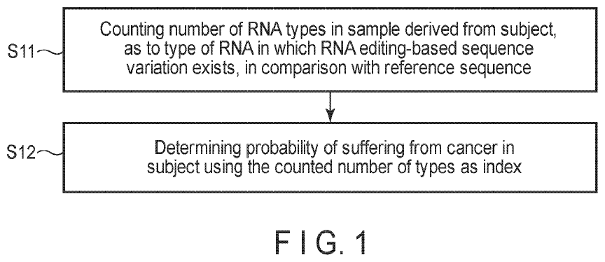

The first embodiment will be described with reference to FIG. 1. This embodiment is a method for analyzing the probability of suffering from cancer in a subject. The analytic method includes counting the number of RNA types in the sample derived from the subject, that is, the number of types, for which sequence variation caused by RNA editing exists in comparison with a reference sequence, and determining the probability of the suffering from cancer in the subject using the counted number of types as an index.

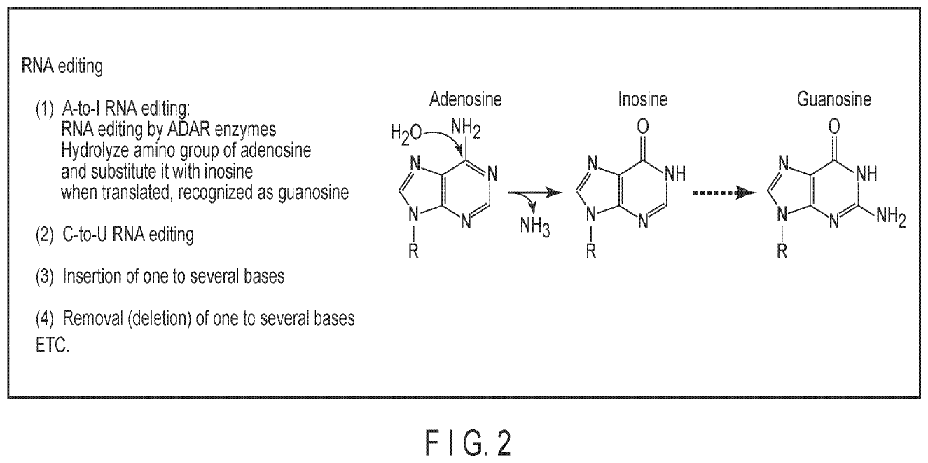

RNA editing is a mechanism in plants and animals that substitutes the sequence of RNA transcribed from DNA or RNA being transcribed, or inserts or deletes one to several bases to the sequence. It has been reported that RNA editing involved in the regulation of various biological processes, and that is considered to be one type of modification mechanism after RNA transcription. With reference to FIG. 2, a typical example of RNA editing will be described. That is, typical examples of RNA editing are, for example, (1) A-to-I RNA editing, (2) C-to-U RNA editing, (3) insertion of one to several bases, and (4) removal (deletion) of one to several bases. A-to-I RNA editing (adenosine to inosine editing) is RNA editing by ADAR enzymes. In A-to-I RNA editing, the amino group of adenosine (A) is hydrolyzed, and then adenosine (A) is substituted for inosine (I). Inosine (I) is recognized as guanosine (G) during translation, which has a similar chemical structure. C-to-U RNA editing (cytidine to uridine editing) is the substitution of cytidine (C) for uridine (U). In the analytical method, regardless of the mechanism of RNA editing, it suffices only if the number of variants in the sample is counted for RNAs with sequence variation in light of the reference sequence.

The reference sequence can be any sequence in which sequence variation has not occurred due to RNA editing, for example, it can be the corresponding wild-type sequence. The information on the wild-type sequence can be obtained from or referred to a gene database for the type of RNA (for example, a database such as Ensembl or NCBI, which collect all gene data, or a database that collects information on a specific type of RNA, such as miRBase, which is a database for miRNAs). When there is an update in the gene database information, the latest information can be referred to as desired. Further, the reference sequence may be a specific full-length RNA, a partial sequence of a specific RNA, or a combination thereof.

Samples derived from a subject includes cells, tissues and/or fluids collected from the subject, or a mixture thereof, or a processed product obtained by appropriate treatment thereof. If the sample is a body fluid, for example, it may be serum or plasma, or it may be other body fluids, such as blood, leukocyte interstitial fluid, urine, stool, sweat, saliva, oral mucosa, nasal mucosa, nasal fluid, pharyngeal mucosa, sputum, digestive fluid, gastric fluid, lymph fluid, spinal fluid, tear fluid, breast milk, amniotic fluid, semen, vaginal fluid or a mixture thereof. Alternatively, the sample may be tissue or cells or a mixture thereof. The sample may as well be one immediately after collection from the subject, cultured, stored in a desired procedure, or the supernatant thereof obtained after they are maintained in a desired liquid. It is preferred to use body fluids such as blood, serum and plasma as samples derived from subject because they are easy to collect.

Information regarding the RNA about the sample may be obtained after the RNA has been extracted from the sample. The method of RNA extraction may be performed by any methods known per se, for example, commercially available kits may as well be used.

The subject is an animal to be analyzed in the method, that is, the animal which provides the sample. The subject may be an animal with some sort of disease or a healthy animal. For example, the subject may be an animal that may have cancer or has had cancer in the past, etc. In particular, the subject may be an animal that may have breast cancer or has had breast cancer in the past. It is preferred that the subject be a human.

Alternatively, the subject may be some other animal. This animal may be of, for example, mammals, including primates such as monkeys, rodents such as mice, rats or guinea pigs, companion animals such as dogs, cats or rabbits, domestic animals such as horses, cows or pigs, or animals belonging to exhibition animals, etc.

In the analytic method, the number of types of RNAs among the population RNA contained in the sample derived from the subject is counted to determine how many types of RNAs have sequence variation caused by RNA editing, and the number of types counted is used as an index to determine the probability of suffering from cancer in the subject. In this analytic method, the expression amount and the number of copies of a specific RNA species are not measured for RNAs having sequence variation, but the number of types of RNAs in the sample from the subject that have a sequence different from the reference sequence due to RNA editing is counted (that is, how many types of RNAs have sequence variation is determined). Although the details will be described later, for example, as to the counting of the number of types, a specific category of RNAs may be exhaustively examined and counted. Or, a specific or arbitrary RNA population may be established, and the number of types may be exhaustively examined and counted for those RNAs. For example, the number of RNA molecules contained in an RNA population may be, for example, the number registered in the database used or arbitrarily selected from it. For example, miRBase at the current time, contains 38589 pre-miRNAs and 4860 miRNAs registered for 271 biological species. Of these, 1917 pre-miRNAs and 2654 miRNAs are registered for the human genome. Any of these numbers could be used as the number of RNA molecules in an RNA population, but it is not limited to these numbers. For example, in the case where a specific RNA population is formed, for example, as to the method is to limit the number of miRNAs to be targeted, such criteria may be considered, as simply selecting from those with high expression levels, selecting from those with small variation in expression levels among samples, selecting from those with a high ratio of mutations, selecting from those with small variation in the ratio of mutations among samples, but not limited to those. Further, the length of RNA can be, for example, from about 17 to 25 bases for miRNAs, but is not limited to these lengths. Or, for pre-miRNAs, for example, it can be about 60 to 70 base pairs.

The RNA to be counted can be any RNA species for which there can be sequence variation caused by RNA editing, such as RNA, mRNA, ncRNA (non-coding RNA), housekeeping ncRNA, tRNA, small ncRNA, miRNA, piRNA, tsRNA, IncRNA and so on. These RNAs may be counted category-inclusive, counted in a mixture of several categories, or counted for a specific category of RNA. For example, when counting a specific category of RNA, it may be mRNA, tRNA, small ncRNA, miRNA, piRNA, tsRNA, etc., or it may be a mixture of at least two of those categories. Alternatively, such a particular category of RNAs may be, for example, miRNAs. For RNAs with sequence variation, rather than the expression level or the number of copies of a particular RNA type, but the number of RNA types distributed in a group of RNAs, can be counted, and the probability of suffering from cancer in the subject can be determined based on the number of types obtained.

In order to more accurately track the sequence variation due to RNA editing, a highly conserved sequence, or a sequence in a highly conserved site, or a sequence containing such may be selected. “A highly conserved sequence, or a sequence in a highly conserved site, or a sequence containing such” means, for example, a sequence of site where the possible sequence variation and diversity (such as polymorphisms, mutations, substitutions, deletions and insertions) in pre-transcribed nucleic acids (for example, genomes) are less frequent and less likely to affect. For example, in the case of miRNAs, the 5′-end positions 1 to 10, which are called seed sequences, generally have low probability of presence of single nucleotide polymorphisms and low sequence variability, and such sequences are considered to be highly useful.

Analyzing the probability of suffering from cancer in a subject may be, for example, to determine whether the subject may or may not have cancer, to determine that the subject has a high or low probability of having cancer, to identify whether the subject is a cancer patient or a non-cancer patient, etc. According to the first embodiment, it is possible to mechanically and/or automatically determine the probability that the subject has cancer based on objective comparative criteria. For example, the method of analyzing the probability of suffering from cancer in the subject can be rephrased as, for example, “a method of obtaining information about the probability of suffering from cancer in the subject”. The obtained information can be used, for example, by a physician to determine, that is, diagnose, a medical condition or health status of a human “subject” for medical purposes. If such “judgment” or “diagnosis” by a physician is “determination of whether or not the target cancer group is affected”, it can be said that the embodiment is an “analytic method to assist” the “physician” in such “determination of whether or not the target cancer group is affected” for medical purposes.

For example, the determination can be made that the subject is likely to have cancer when the number of types is greater than a predetermined threshold value. Alternatively, the determination can be made that the subject is unlikely to have cancer if the number of types is less than the predetermined threshold value. The threshold value may be determined and set by comparing the results obtained using samples derived from non-cancer patients with those obtained using samples derived from typical cancer patients, with corresponding the population RNA and reference sequences.

In this specification, the term “cancer” covers any stage thereof, including, for example, a state in which the cancer remains in the organ of origin, a state in which the cancer has spread to surrounding tissues, a state in which the cancer has metastasized to lymph nodes, and a state in which the cancer has metastasized to further distant organs. In this specification, the term “breast cancer” refers to a malignant tumor (neoplasm) that forms in the mammary gland tissue. For example, the term “breast cancer” includes what is commonly referred to as “breast cancer” or “breast carcinoma”. Further, the term “breast cancer” according to the embodiment also includes any type of breast cancer, for example, lobular carcinoma of the mammary gland or ductal carcinoma of the mammary gland. Furthermore, the breast cancer according to embodiments also includes, for example, epithelial tumors, non-epithelial tumors, and malignant lobular tumors composed of both epithelial and non-epithelial natures.

For example, the term “cancer” can be at least one cancer selected from the group consisting of breast cancer, colorectal cancer, lung cancer, stomach cancer, pancreatic cancer, cervical cancer, uterine cancer, ovarian cancer, sarcoma, prostate cancer, bile duct cancer, bladder cancer, esophagus cancer, liver cancer, brain tumor, and kidney cancer.

The method of the embodiment is effective in time and economic efficiency of clinical development because it is a qualitative test, not a quantitative test, and there is no need to ensure quantitative performance.

For example, serum or plasma, which can be easily collected during medical examinations or the like, can be used as a sample. Therefore, for example, with use of serum collected from the subject, it is possible to detect cancer comprehensively, that is, universally, as a primary screening for cancer at the time of health checkups. Thus, cancer can be detected at an early stage. By using serum or plasma, etc., the physical and economic burden on the subject can be greatly reduced compared to cytological diagnosis, etc., and the procedure is easy and less burdensome for the examiner.

### Second Embodiment

The second embodiment is a method for analyzing the probability of suffering from cancer in the subject. In this analytic method, in addition to counting the number of types for samples from the subject as presented in the first embodiment, the number of types for samples from the control, for example, a non-cancer control is also counted. Using the counted number of types as an index, the probability of suffering from cancer in the subject is determined by comparing the number of types from the subject with the number of types from the non-cancer control in this embodiment.

The control can be, for example, a healthy subject. The healthy subject can be at least an individual who does not have cancer. The healthy subject should preferably be a healthy individual without disease or abnormality. The individual selected as a control may be an individual different from the subject to be analyzed by the method, and should preferably be an individual belonging to the same species, that is, a human if the subject is a human. Further, the physical condition such as age, sex, height, weight or the like or the number of individuals of the control are not particularly limited, but the physical condition should preferably be the same or similar to that of the subject to be examined in this analytic method. Alternatively, a sample derived from the subject may be taken over time and the test results of the subject when healthy subject may be used as a control, non-cancer individual or non-cancer control.

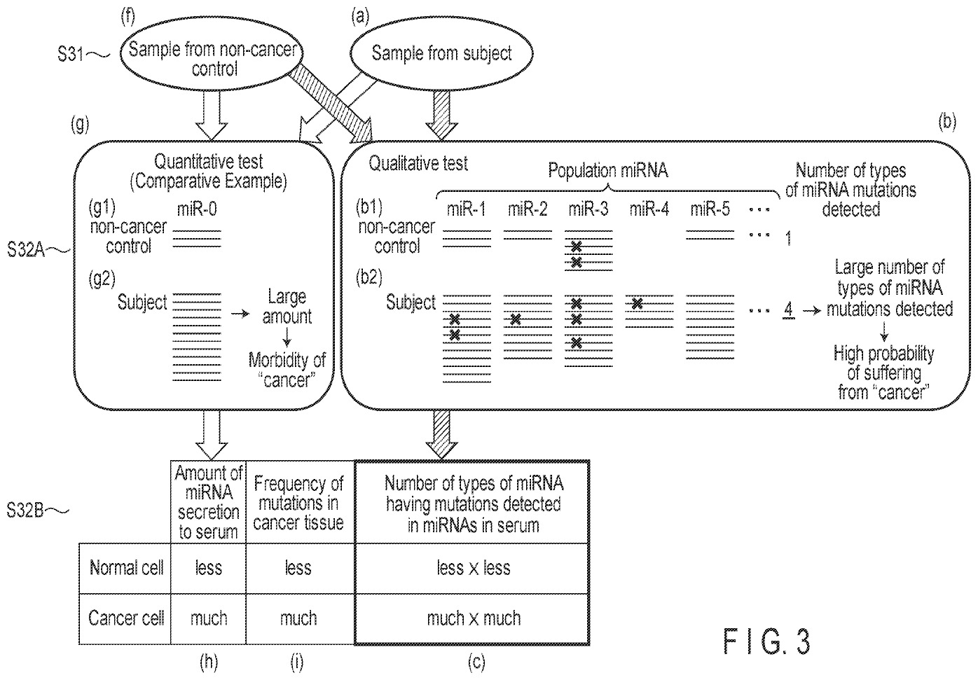

With reference to FIG. 3, one example of such analytic method as the second embodiment will be conceptually explained. As one example of the second embodiment, such a case will be presented, where the subject is a human, and miRNAs are selected and counted as a specific category of RNAs (FIG. 3, S31(a), S32A, and S32B). In FIG. 3, “subject” represents an individual as the subject of this method, “control” represents a non-cancer control, and “population RNA” represents a population miRNA. FIG. 3 further conceptually shows one example of a general quantification test as a comparative example (FIG. 3, S31(f), S32A(g) and S32B(h)).

First, a sample (a) from the subject and a sample (f) from a non-cancer control are prepared (S31). Using the specific categories of RNAs contained in these samples as the population, the number of types in the samples derived from the subject patient and non-cancer control is counted with respect to the types of RNAs for which sequence variation caused by RNA editing is present (S32A (b), (b1) and (b2)). In this example, such a case is presented that miRNAs present in serum were comprehensively classified and analyzed as the population miRNA, and the types of RNAs for which sequence variation caused by RNA editing is present were counted. Five types of the population miRNA, namely, miR-1, miR-2, miR-3, miR-4, and miR-5, are shown in FIG. 3 as representative fractions, and the other fractions are omitted (S32A(b)). The criterion in this case is that the individuals as the subjects are identified as likely to have cancer if RNA editing-caused sequence variation is present in more types of miRNAs than that in the non-cancer control.

For convenience, the number of copies is referred to in the following simulations. In the sample of the non-cancer subject, it is assumed that there were 3 copies of miR-1, 2 copies of miR-2, 6 copies of miR-3, 0 copy of miR-4, and 2 copies of miR-5 and they are secreted into the serum (S32A(b1)). RNA editing was observed in 2 copies of miR-3 among them (each marked with x). In this case, there is only one RNA, namely miR-3, for which there is sequence variation caused by RNA editing in the sample from the non-cancer subject. By contrast, the results of the simulation are presented below, assuming that the subject was a cancer patient. In the subject, there were 12 copies of miR-1 secreted into the serum, 9 copies of miR-2, 10 copies of miR-3, 5 copies of miR-4, and 9 copies of miR-5 (S32A(b2)). Among them, RNA editing was observed in 2 copies of miR-1, 1 copy of miR-2, 3 copies of miR-3, and 1 copy of miR-4 (each marked with x). Thus, there are four RNAs, miR-1 to miR-4, for which there are sequence variations caused by RNA editing in the subject-derived samples. By comparison, the subject has four types, compared to one type in the control, involving a larger number of types. This result identifies the subject as likely to have cancer.

This analytical method is a qualitative test, which simply determines the presence or absence of mutations caused by RNA editing and further, by comparing the number of such types between a non-cancer control and the subject, determines the probability of a subject having “cancer”, without any consideration of the sequence, target, function, phenotype, or other characteristics of each type of miRNA in the population miRNA used here.

By contrast, in the case where a generally known specific type of miRNA is used as a cancer marker to determine the high or low probability of “cancer” of the subject, a quantitative test is conducted. That is, as shown in FIG. 3, part (g), a sample (a) from the subject and a sample (f) from a non-cancer control are prepared (S31), and then the expression level (S32A (g1)) of a specific miRNA (shown here as miR-0 for convenience) in the sample from the non-cancer subject and the expression level (S32A (g2)) of the corresponding miRNA (here, miR-0) in the sample from the subject are compared with each other. Then, as a result of the comparison, for example, when the miR-0 in the sample from the subject is higher in its amount than in the control, the subject is determined to be likely to have “cancer”.

In the analytic method of this embodiment, it can be said that the counting of the number of RNA types, in which sequence variation caused by RNA editing is present, covers information on both the mutation frequency in cancer tissue and the amount of miRNA secreted into the serum, as shown in S32B, for example. In other words, in general, in the case of quantitative tests, for example, the information that the amount of miRNA secreted into the serum is low in normal cells and high in cancer cells (S32B(h)) and the information that the mutation frequency in cancer tissue is low in normal cells and high in cancer cells (S32B(i)) are multiplied together to obtain such information that the number of types of miRNAs in which mutations are detected in the miRNAs of the serum is (less×less) in normal cells and (more×more) in cancer cells (S32B (c)). In this manner, the results are represented in an emphasized manner and more easily comprehensible. In addition, the test can be of a qualitative one using the number thereof as the indicator rather than the concentration.

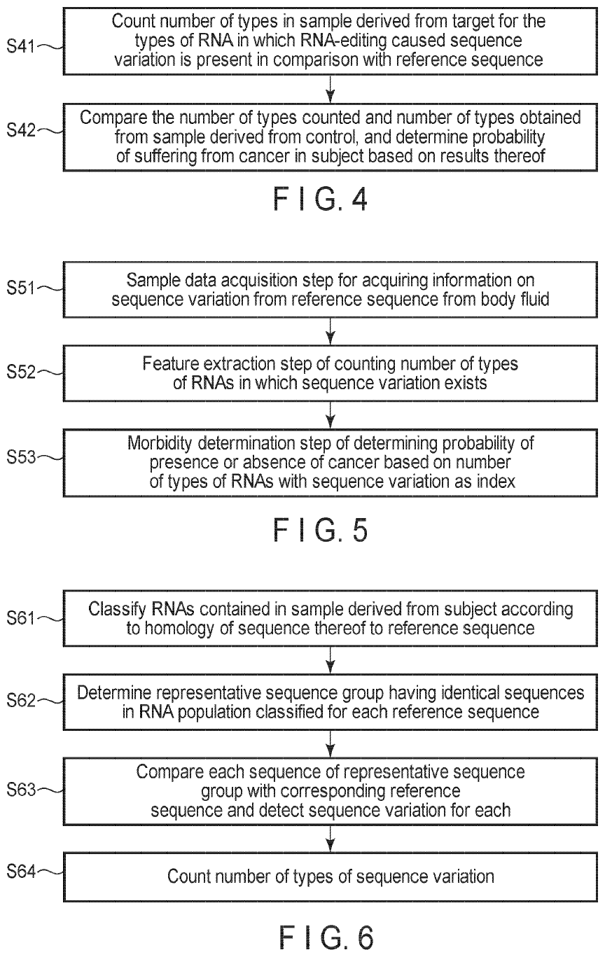

The second embodiment as described above is shown in the scheme diagram in FIG. 4, may include counting the number of types in the sample derived from the target for the types of RNA in which sequence variation caused by RNA variants is present, compared to the reference sequence (S41), and comparing the number of types obtained and the number of types obtained from the sample derived from the control to determine the probability of suffering from cancer in the subject based on the results of the comparison (S42).

The miRNAs to be the population may be arbitrarily selected, designed and/or set from any type of miRNAs contained in any database of miRNAs known per se, or after creating their own database using a known discovery tool, etc. Examples of miRNA databases may be miRNABase, Rfam, miRIAD, dbDEMC, etc. Examples of discovery tools may be miRscan, miRNAFold, miRDeep, miRanalyzer, ChIPBase, sRNAbench, etc. Alternatively, they may be arbitrarily selected from miRNA databases where sequences that can cause mutations due to RNA editing, such as MiREDiBase (miRNA Editing Database) (Sci Data. 2021 Aug 4; 8(1):199. PMID: 34349127), are registered. The number of miRNAs in the population may be, for example, 300, 400, 500, or 600 types or even more.

The second embodiment is based on the findings of the inventors that nucleic acids can be extracted from body fluids, which can be collected minimally invasively, and that sequence variation can be used to discriminate healthy individuals from those affected by cancer. In other words, according to the second embodiment, it is possible to extracted nucleic acids from body fluids, which can be collected minimally invasively, and to discriminate between healthy individuals and cancer patients using the sequence variation. In addition, the test is a qualitative test rather than a quantitative test and therefore it can eliminates the need to ensure quantitative performance, making it superior in terms of time and economic efficiency in clinical development. Further, for example, using serum collected from the subject, it is possible to detect cancer comprehensively, that is, universally, as a primary screening for cancer at the time of physical checkup.

### Third Embodiment

FIG. 5 shows the third embodiment, which is directed to a method for analyzing the probability of suffering from cancer in a subject. The method comprises three processes, namely, a sample data acquisition step (S51), a feature extraction step (S52), and a morbidity determination step (S53). In the sample data acquisition step, information on sequence variation from a reference sequence is obtained from body fluids collected from the subject (S51). The information regarding sequence variation from the reference sequence is information on RNA contained in the body fluid. In the feature extraction step, the number of types of RNAs for which sequence variation exists is counted (S52). In the morbidity determination step, the number of types of RNAs with sequence variation is used as an index to determine the probability of whether or not the patient has cancer. Alternatively, the morbidity determination step may be a step in which the subject is identified as a cancer patient or a non-cancer patient using the indicator.

Since the test is a qualitative test rather than a quantitative test and therefore it can eliminates the need to ensure quantitative performance, making it superior in terms of time and economic efficiency in clinical development.

For example, serum or plasma, which can be easily collected during medical checkups or the like, can be used as a sample. Therefore, for example, it is possible to detect cancer comprehensively, that is, universally, as a primary screening for cancer at the time of health checkups, using serum collected from the subject. Thus, it is possible to detect cancer in an early stage. By using serum or plasma or the like, the physical and economic burden on the subject can be greatly reduced compared to cytological diagnosis, etc., and the procedure is easy and less burdensome for the examiner. In addition, serum or plasma has a stable concentration of RNA contained therein, for example, miRNA concentration, and therefore even more accurate testing can be performed.

### Fourth Embodiment

FIG. 6 shows the fourth embodiment, which is directed to a method for analyzing the probability of suffering from cancer in a subject. The method comprises four processes. First, RNAs contained in a sample derived from the subject are classified according to the homology of the sequence to a reference sequence (S61). Next, a representative sequence group having identical sequences in the RNA population classified for each reference sequence is determined (S62). Each sequence of the representative sequence group is compared with the corresponding reference sequence to detect sequence variation for each (S63). Further, the number of types of such representative sequences with sequence variation is counted (S64). This method can as well be used as a method for detecting sequence variation. The method for detecting sequence variation can comprise: classifying RNAs contained in a sample derived from a subject according to homology of the sequences to a reference sequence; determining a group of representative sequences having identical sequences in the RNA population classified for each reference sequence; comparing each sequence in the representative sequence group with the corresponding reference sequence, and detecting sequence variation. Furthermore, it can as well be a method for detecting sequence variation wherein in a particular representative sequence, sequence variation in comparison with the corresponding reference sequence can be identified together with sequence information of the representative sequence of interest.

The classification by homology of RNA sequences means that a plurality of groups are established by classifying sequences that are highly homologous to each other into one group with respect to the sequence of a specific site of RNA for which information is to be obtained. In doing so, a reference sequence (a known sequence obtained from a database) is used as the standard sequence for the group. The classification by homology can be done, for example, by using next-generation sequencing (NGS), qPCR, Sanger sequencing, microarrays for RNA detection, hybridization or the like. Or it may be done by using a combination of at least two of these techniques.

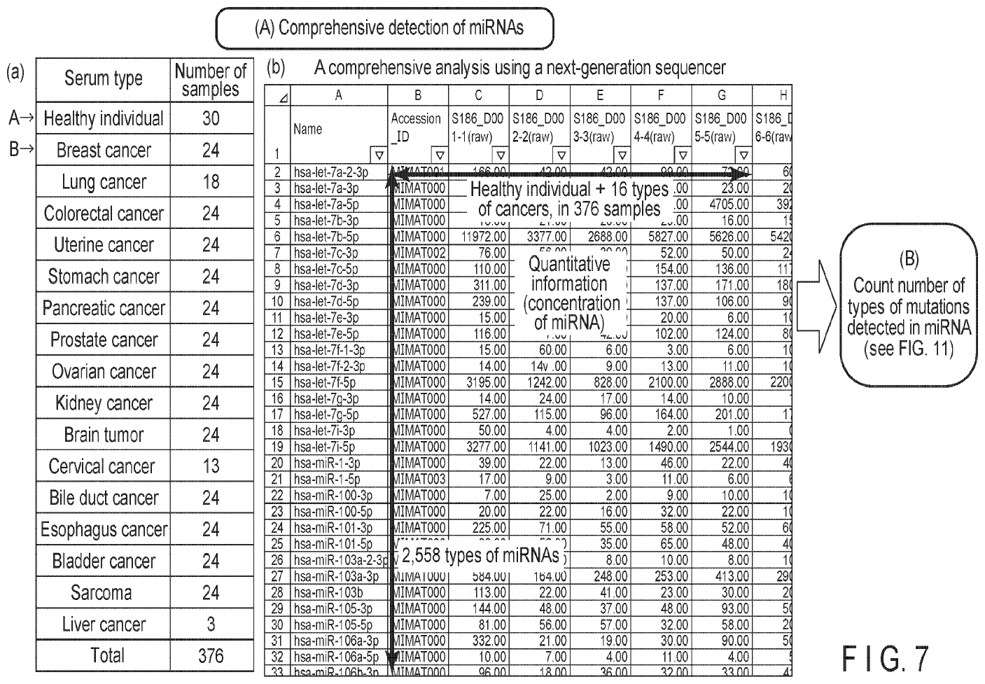

FIGS. 7 to 11 show images of an example of a comprehensive analysis of miRNAs using a next-generation sequencer. As shown in FIG. 7(a), as the samples, serums were used, which were collected from 30 healthy subjects, 24 breast cancer cases, 18 lung cancer cases, 24 colorectal cancer cases, 24 uterine cancer cases, 24 stomach cancer cases, 24 pancreatic cancer cases, 24 prostate cancer cases, 24 ovarian cancer cases, 24 kidney cancer cases, 24 brain tumor cases, 13 cervical cancer cases, 24 bile duct cancer cases, 24 esophagus cancer cases, 24 bladder cancer cases, 24 sarcoma cases and 3 liver cancer cases. As shown in FIG. 7, part (b), 2,654 miRNAs were analyzed, which include hsa-let-7a-2-3p, hsa-let-7a-3p, hsa-let-7a-5p, hsa-let-7b-3p, hsa-let-7b-5p, hsa-let-7c-3p, hsa-let-7c-5p, hsa-let-7d-3p, hsa-let-7d-5p, hsa-let-7e-3p, hsa-let-7e-5p, hsa-let-7f-3p, hsa-let-7f-5p, hsa-let-7g-3p, hsa-let-7g-5p, hsa-let-7i-3p, hsa-let-7i-5p, hsa-miR-100-3p, hsa-miR-100-5p, hsa-miR-101-3p, hsa-miR-101-5p, hsa-miR-103a-2p, hsa-miR-103a-3p, hsa-miR-103b, hsa-miR-105-3p, hsa-miR-103-5p, hsa-miR-106a-3p, hsa-miR-106a-5p and hsa-miR-106b-3p. First, these miRNAs were quantified by next-generation sequencing in one case each for healthy subjects and cancer patients. FIG. 7(b) shows some of the results, with the names of the miRNAs in column A, and the 30 cases of healthy subjects and each cancer affected person data in columns C, D, E . . . , in order. That is, the RNAs contained in the samples derived from the subject are classified according to their sequence homology.

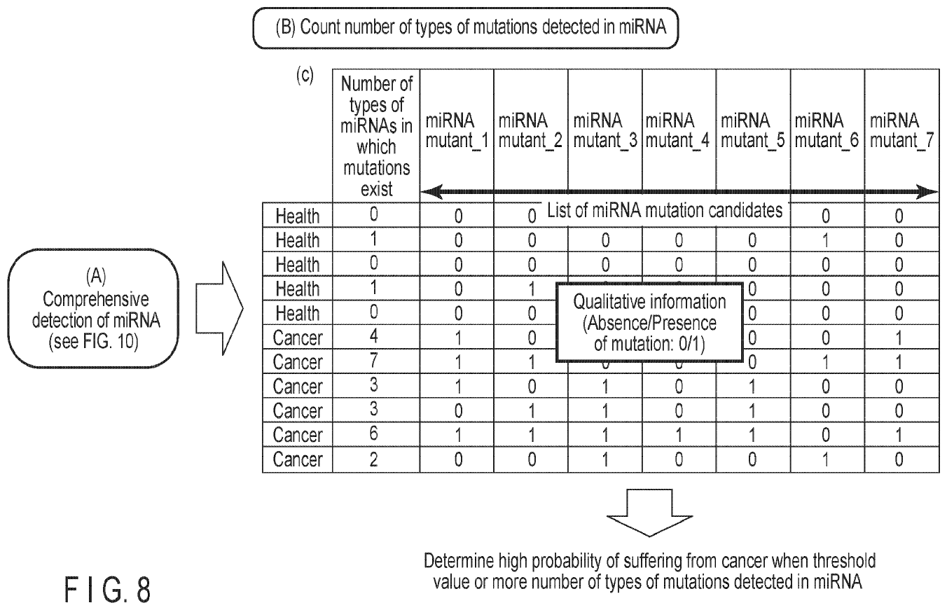

Next, in order to determine whether each of the classified populations (i.e., each miRNA, for example AGCTAGCT) involves sequence variation compared to the reference sequence, one or a plurality of sequences involving sequence variation compared to the reference sequence is selected among a representative sequence group (i.e., AGCTAGCT (wild type) or mutant AGCTGGCT or AGCTAGTT) of each of the classified populations. The sequences with sequence variation selected from each population were designated as the candidate list. That is, the candidate list consists of the name of the population and the sequence information of the sequences with sequence variation. Here, when there is a sequence identical to the sequence with the sequence variation (=AGCTGGCT or AGCTAGTT) selected to be identical to that the population of the candidate list, it was determined to have a mutation as compared to the candidate. The table in FIG. 8, part (c) is part of the data from FIG. 7, part (b), where the presence or absence of mutations was determined for the types of miRNAs in the pre-generated miRNA mutation candidate list for five healthy subjects and six cancer-affected subjects. In FIG. 8, part (c), miRNA_mutant_1, miRNA_mutant_2, miRNA_mutant_3, miRNA_mutant_4, miRNA_mutant_5, miRNA_mutant_6, and miRNA_mutant_7 are shown as part of the candidates of those of miRNA mutations. The ones with mutations were entered as “1”, and those without mutations were entered as “0”, and the number of miRNA types for which mutations were present in the entire mutation list was counted. When the number of types of miRNA mutations detected was at a predetermined threshold or a threshold determined from time to time, or more, it is determined that the person is likely to be a cancer patient.

The determination of the threshold value may be performed by analyzing and comparing samples derived from cancer patients and samples derived from non-cancer patients in advance, or by analyzing and comparing information from non-cancer patients each time samples derived from the subject are analyzed. For example, examples of threshold values are 2, 3, 5, 10, 23, 25, 26, 30 or the like, but are not limited to these. The number of types of miRNA to be used as markers may be set as the maximum value. For example, the threshold may be determined by the method and the type of RNA used in the analytical method.

The method is a qualitative test, rather than a quantitative test, and therefore it can eliminate the need to ensure quantitative performance, which is excellent for time and economic efficiency in clinical development. Further, it is possible to detect cancer comprehensively, that is, universally, as a primary screening for cancer at the time of medical checkups, for example, using serums collected from the subject.

For example, serum or plasma that can be easily collected during medical checkups or the like can be used as a sample. Therefore, cancer can be detected at an early stage. Further, by using serum or plasma, the physical and economic burden on the subject can be greatly reduced compared to cytological diagnosis, etc., and the procedure is easy and less burdensome for the examiner. In addition, serum or plasma has a stable concentration of RNA contained therein, for example, the miRNA concentration, and therefore more accurate testing can be performed.

### Fifth Embodiment

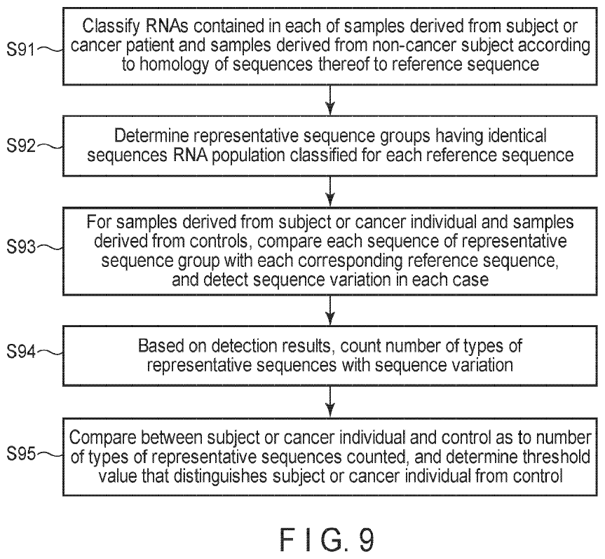

The fifth embodiment is directed to a method for setting a threshold value used to determine that a person is likely to be a cancer patient. The method for setting the threshold comprises four processes (FIG. 9). First, for samples derived from the subject or cancer patient and samples derived from non-cancer patients, the RNAs contained in each are classified according to the homology of their sequences to the reference sequence (S91). Next, representative sequence groups having the same sequence are determined in the RNA population classified for each reference sequence (S92). As to the samples derived from the subject or cancer patient and the samples derived from controls, each sequence of the representative sequence group is compared with each corresponding reference sequence, and thus sequence variation is detected in each case (S93). Based on the detection results, the number of types of representative sequences with sequence variation is counted (S94). The number of types of representative sequences counted is compared between the subject or cancer patient and the control, and a threshold value that distinguishes the subject or cancer patient from the control is determined (S95).

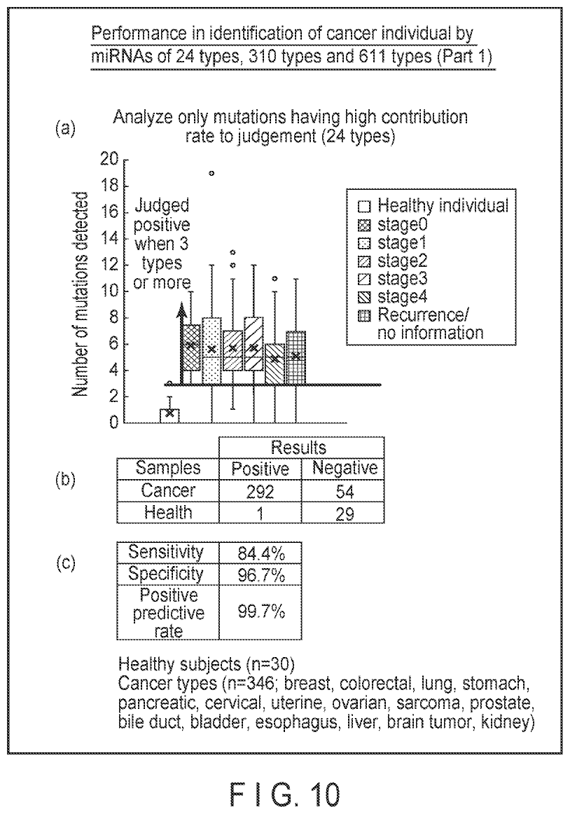

The method of setting the threshold value is shown in FIGS. 10 to 12 as an image of setting the threshold value for the case where miRNA is designated as one example of RNA. FIG. 10 shows an example of comparing samples derived from cancer patients and those of healthy persons as controls for 24 types of miRNAs. In this example, the number of healthy subjects was 30 (n=30). The number of cancer patients was 346 (n=346), and the breakdown in cancer types included 24 breast cancer cases, 18 lung cancer cases, 24 colorectal cancer cases, 24 uterine cancer cases, 24 stomach cancer cases, 24 pancreatic cancer cases, 24 prostate cancer cases, 24 ovarian cancer cases, 24 kidney cancer cases, 24 brain tumor cases, 13 cervical cancer cases, 24 bile duct cancer cases, 24 esophagus cancer cases, 24 bladder cancer cases, 24 sarcoma cases and 3 liver cancer cases.

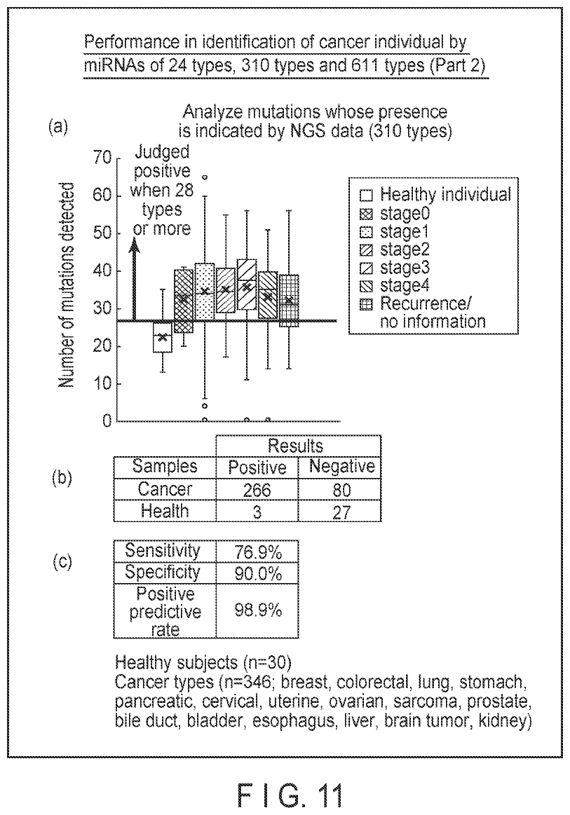

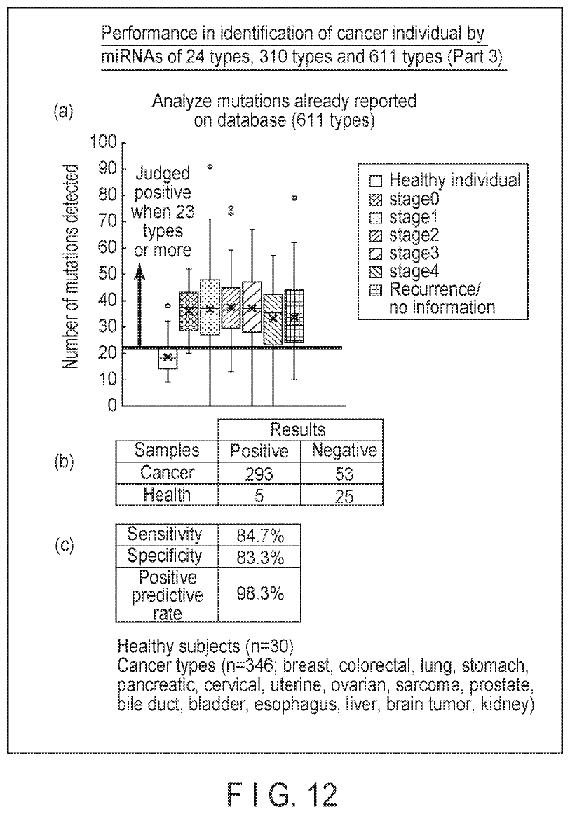

FIG. 10, part (a), FIG. 11, part (a) and FIG. 12, part (a) are each a graph showing the types of representative sequences in controls and the number of representative sequences in cancer patients. More specifically, the number of representative sequences with edit-based sequence variation in miRNAs derived from healthy individuals and the number of representative sequences with edit-based sequence variation in miRNAs derived from cancer-affected patents are shown. The number of representative sequences derived from the cancer patients is shown for each of cancer stages 0, 1, 2, 3, and 4. Further, the data for cancer patients without recurrence information is also presented.

In FIG. 10, 24 types of miRNAs that were considered to be mutations with high contribution to the determination of the presence or absence of cancer incidence were used as representative sequences. In FIG. 11, 310 types of miRNAs that showed the presence of mutations based on RNA editing in NGS data were used as representative sequences. In FIG. 12, 611 types of miRNAs with RNA editing-based mutations already reported in the database were used as representative sequences. For each case, the number of representative sequences with sequence variation in miRNAs from healthy individuals and miRNAs from cancer patients are plotted in FIG. 10, part (a), FIG. 11, part (a) and FIG. 12, part (a).

As shown in FIG. 10, when 24 representative sequences are used, with regard to the number of representative sequences with sequence variation, that is, the number of mutation detections (vertical axis of the graph), the threshold value by which healthy subjects and cancer patients are separated from each other, can be set to 3. FIG. 10, part (b) shows the results which verify that the threshold value of 3 is effective. In the verification, the threshold value was set to 3, and the presence or absence of suffering from cancer was determined for all cancer patients and all healthy subjects in an actual test. In the cancer patient group, 292 samples were determined to be positive and 54 samples were determined to be negative.

In the healthy subject group, one sample was determined to be positive and 29 samples were determined to be negative. As shown in FIG. 10, part (c), in this setting, the performance of the method for analyzing the probability of suffering from cancer in the subjects was 84.4% in sensitivity, 96.7% in specificity, and 99.7% in positive predictive rate, which clearly verified that the method is worthy of practical use.

As shown in FIG. 11, when 310 representative sequences are used, with regard to the number of representative sequences with sequence variation, that is, the number of mutation detections (vertical axis of the graph), the threshold value by which healthy subjects and cancer patients are separated from each other, can be set to 28. FIG. 11, part (b) shows the results which verify that the threshold value of 28 is effective. In the verification, the threshold value was set to 28, and the presence or absence of suffering from cancer was determined for all cancer patients and all healthy subjects in an actual test. In the cancer patient group, 266 samples were determined to be positive and 80 samples were determined to be negative. In the healthy subject group, 3 samples were determined to be positive and 27 samples were determined to be negative. As shown in FIG. 11, part (c), in this setting, the performance of the method for analyzing the probability of suffering from cancer in the subjects was 76.9% in sensitivity, 90.0% in specificity, and 98.9% in positive predictive rate, which clearly verified that the method is worthy of practical use.

As shown in FIG. 12, when 611 representative sequences are used, with regard to the number of representative sequences with sequence variation, that is, the number of mutation detections (vertical axis of the graph), the threshold value by which healthy subjects and cancer patients are separated from each other, can be set to 23. FIG. 12, part (b) shows the results which verify that the threshold value of 23 is effective. In the verification, the threshold value was set to 23, and the presence or absence of suffering from cancer was determined for all cancer patients and all healthy subjects in an actual test. In the cancer patient group, 293 samples were determined to be positive and 53 samples were determined to be negative. In the healthy subject group, 5 samples were determined to be positive and 25 samples were determined to be negative. As shown in FIG. 12, part (c), in this setting, the performance of the method for analyzing the probability of suffering from cancer in the subjects was 84.7% in sensitivity, 83.3% in specificity, and 98.3% in positive predictive rate, which clearly verified that the method is worthy of practical use.

According to the fifth embodiment, it is possible to provide a threshold value that can be used in the method for analyzing the probability of cancer suffering from the subject in the first to fourth embodiments.

### Sixth Embodiment

As a marker for discriminating between healthy subjects and cancer-affected subjects, sequence variation caused by RNA editing can be used. The marker of the sixth embodiment is directed to the number of RNA types in the sample derived from the subject for the type of RNA in which RNA editing-caused sequence variation exists compared to the reference sequence. In other words, it is the number of representative sequences with sequence variation in the sample derived from the subject.

The RNAs to be detected in the serum can be, for example, miRNAs, but there is no restriction on the number and combination of miRNAs. For the sequence variation of the selected miRNA, the number of types of sequence variation that occurred in the examinee i.e., the subject, is counted, and the number is used to discriminate between healthy subjects and those affected by cancer.

For a cause of sequence variation, A to G mutation by the A-to-I RNA editing enzyme ADAR1 can be listed as a candidate. ADAR1 is an enzyme that converts adenosine (A) in double-stranded RNA to inosine by a hydrolytic deamination reaction. Here, because the structure of inosine is similar to that of guanosine (G), it is recognized as guanosine during translation. In other words, it will have a phenotype equivalent to that of an A to G mutation in terms of gene sequence. Alternatively, C to U conversions are also known, but are not limited to these. It is also considered to limit the position on the gene where the sequence variation occurs to a specific site. Here, with respect to the location of SNPs that have been shown to have diversity, for example, among the racial groups to be tested as SNPs, it is known in advance that a certain percentage of the tested population has a variation, and therefore such a restrictive method can be considered that a variation is not considered as sequence variation caused by cancer. In other cases, when the regions where sequence variation may occur or does not easily occur are known biologically, the regions of particular interest may be limited according to the probability of occurrence and the reasons for it, but it is not limited thereto.

The process of determining the number of sequence variations of miRNAs in the body fluids should preferably be constituted mainly by: (i) collecting samples from the subject; (ii) extracting miRNAs from the samples; (iii) decoding the sequences of target miRNAs; (iV) classifying the sequences according to the types of miRNAs; and (V) detecting the presence or absence of sequence variations within each type of miRNA, and representative methods will be described below, but are not limited to these. Further, subsequently, the morbidity determining step of discriminating the presence or absence of suffering from cancer based on the number of sequence variations should preferably be constituted by: (VI) setting a threshold value; and (VII) comparing the value with the threshold value whether it is larger or smaller, and representative methods of which will be described below, but not limited to these.

### Sample Collection from Subject

Samples used for the measurement are those collected from the subject and are not limited to any particular sample, but, for example, blood, serum, plasma, white blood cells, urine, digestive fluids, saliva, gastric juices, sweat, tears, nasal fluid, semen, vaginal fluid, amniotic fluid, milk, lymph fluid, tissue, oral mucosa, and sputum can be used.

Samples are subjected to treatment such as centrifugation, precipitation, extraction and/or separation to make them into a state suitable for amplification of nucleic acids. Here, if the collected sample is suitable for amplification of nucleic acids as it is, the collected sample may be used as a specimen.

### Extraction of miRNA from the Sample

Nucleic acid extraction can be performed using commercially available nucleic acid extraction kits, including, but not limited to, NucleoSpin (registered trademark) miRNA Plasma (manufactured by Takara Bio), Quick-cfRNA Serum & Plasma Kit (manufactured by Zymo Research), miRNeasy Serum/Plasma Kit (manufactured by Qiagen), miRVana (registered trademark) PARIS isolation kit (manufactured by Thermo Fisher), PureLink (trademark) Total RNA Blood Kit (manufactured by Thermo Fisher), Plasma/Serum RNA Purification Kit (manufactured by Norgen Biotech), microRNA Extractor (registered trademark) SP Kit (manufactured by Wako Pure Chemicals), High Pure miRNA Isolation Kit (manufactured by Sigma-Aldrich), etc. Alternatively, such a simple method can be used, that the sample is diluted with buffer and centrifuged after heat treatment at 80 to 100° C. to obtain the supernatant.

(iii) Decoding the sequences of target miRNAs, (iV) classifying the sequences according to the types of miRNAs, and (V) detecting the presence or absence of sequence variations within each type of miRNA

As one method for measuring the sequence of miRNAs in body fluids, there is a method of comprehensively decoding sequence in miRNAs in body fluids using a next-generation sequencer (NGS). Alternatively, another method which includes amplifying only the target miRNAs using primers that can specifically amplify, and confirming the sequence by the Sanger sequencing can as well be used. But the methods are not limited to these.

When using a next-generation sequencer, the sequencer such as the MiSeq and NextSeq550 manufactured by Illumina, or a single-molecule sequencer manufactured by Pacific Biosciences can be used, but it is not limited thereto. By performing alignment using the human genome sequence as a reference, for example, it is possible to classify sequences according to the type of miRNA. For alignment, BWA, bowtie, bowtie2, etc., can be used, but it is not limited to these.

When using the Sanger sequencing method, primers are designed and sequenced according to the type of miRNA. Alternatively, if the sequences before and after the sequence variation can be determined, primers specific to the sequence after the sequence variation is designed, so that it can be determined that a sequence variation has occurred if the sequence can be decoded with those primers. In such a case, the qPCR or digital PCR methods can be used. Alternatively, a microarray to which a probe specific for sequence variation is added can be used.

(VI) Setting of threshold value, (VII) comparison with threshold value as to greater/smaller

The threshold value may be set, for example, by using a receiver operating characteristic (ROC) curve or by considering the effects of false positives and false negatives. However, the threshold value is not limited to these, and it is considered that the threshold value varies depending on the designing of the test.

The ROC curve is obtained by plotting (1−specificity) on the x-axis and the sensitivity on the y-axis, and in an ideal test (100% sensitivity and 100% specificity), it would be located in the upper left corner. Here, the utility of the test can be evaluated based on the area under the curve ROC. The threshold value is set as follows. In detail, there are two ways to set the threshold value: a method called Youden Index, which selects the threshold that maximizes (sensitivity+specificity), and a method that selects the threshold that minimizes the distance ((1−sensitivity)²+(1−specificity)²) from the upper left corner of the curve ROC. When the threshold is set by using any of these methods, it is generally the case that false positives and false negatives are of equal importance. On the other hand, for example, if the cancer is in an early stage, the therapy for cure has been established, and the type of the cancer affecting the subject which is important to reliably detect the positive subjects, it can be considered more essential to lower the false negatives than the false positives.

In such cases, the threshold can be set by considering a greater weighting of false negatives over false positives. For setting such a threshold, statistical software such as EZR (Bone Marrow Transplantation 2013: 48, 452-458) or JMP can be used.

### Example 1

The discrimination between cancer patients and healthy subjects using the sequence variation of nucleic acids in serum as an index will now be described.

There were 30 serum samples from healthy subjects and 346 samples from cancer patients prepared.

The cancer-affected samples included 24 samples from breast cancer patient, 24 samples from colorectal cancer patient, 24 samples from gastric cancer patient, 18 samples from lung cancer patient, 24 samples from ovarian cancer patient, 24 samples from pancreatic cancer patient, 24 samples from biliary tract cancer patient, 24 samples from esophageal cancer patient, 3 samples from liver cancer patient, 24 samples from brain tumors patient, 24 samples from bladder cancer patient, 24 samples from prostate cancer patient, 24 from sarcoma samples patient, 24 samples from uterine cancer patient, 24 samples from kidney cancer patient, and 13 samples from cervical cancer patient.

The nucleic acid sequences in serum were determined by the next-generation sequencing analysis. The miRNA was extracted from 300 μL of each of all serums using the miRNeasy Serum/Plasma Kit (Qiagen). The extracted miRNAs were handled according to the protocol using QIAseq miRNA Library Kit (Qiagen) and QIAseq miRNA NGS 96 Index IL (Qiagen).

In the indexes used, the molecular bar-code technology called UMI was used. This technology can eliminate the influence by PCR duplication or amplification bias by gene amplification, which may occur in library adjustment, and therefore an even more accurate sequence can be determined. The NGS analysis was performed using NextSeq500 (single-ended, 75 bp), and for all the samples, data of 10,000,000 reads or more was obtained. The extract command of UMI-tools (Genome Res. (2017) 27(3):491-499. PMID: 28100584) was used to obtain a UMI-removed FASTQ file.

Further, QC by read quality was performed. As classification of sequences according to the type of miRNA, annotation was performed with respect to miRBase Release 22. The miRBase Release 22 sequence was designated as wild type, that is, a sequence in which no sequence variation has occurred. As a candidate for sequence variation, especially, 10 bp at the 5′ end of miRNAs was focused. At the 5′ end of miRNAs, about 7 bp, 2-8 bases from the end, there is a region called the Seed sequence.

This region is regarded to be important for miRNAs to perform their functions, so that it is considered to have a low occurrence rate of sequence variation dependent on individual genetic information in the region. As a candidate for sequence variation, A-to-G mutation by ADAR1, A-to-I RNA editing enzyme, was focused. The sequences that can introduce A-to-I mutations to miRNAs are consolidated in the database (MiREDiBase). Here, a list of sequence variations which satisfy the condition was obtained, and it was found that there were 611 types of sequence variation candidates for 388 types of miRNAs. Then, the sequence information of the miRNAs of interest was extracted from the annotation results of the NGS analysis, and each sample was analyzed as to whether or not sequence variations that match those of the database existed.

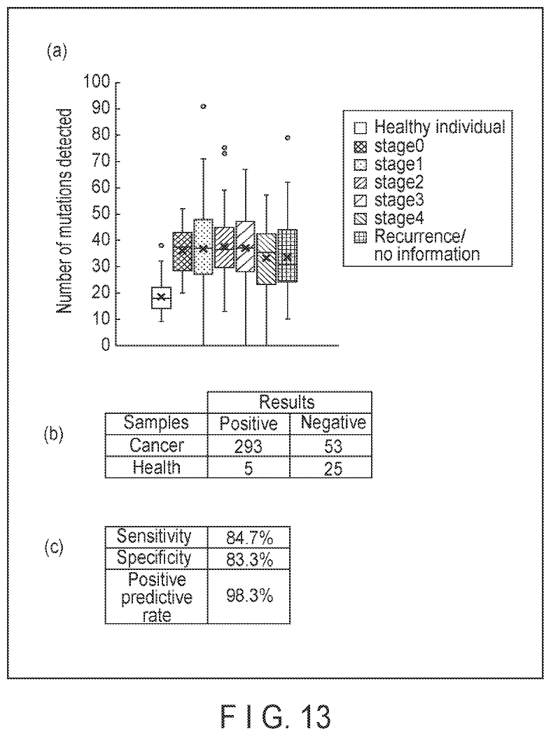

For each sample, out of 611 types of sequence variation candidates, the number of types in which sequence variation occurred was counted, and the total number was used in the subsequent analysis as the number of types of sequence variation in each sample. Then, the ROC curves were plotted using the discrimination (cancer/healthy subject) of each specimen and the number of types of sequence variations, and thus AUC values were calculated. The results are shown in FIG. 13.

The ROC curve is obtained by plotting (1−specificity) on the x-axis and the sensitivity on the y-axis. On the ROC curve, the ideal test result (i.e., 100% sensitivity and 100% specificity) is located at the upper left corner. Here, the utility of the test can be evaluated based on AUC, the area under the ROC curve. The results indicate that the AUC value was 0.887 (95% confidence interval: 0.841-0.934). The threshold was set so that a subject was determined to have cancer if sequence variation was confirmed in 23 or more of the 611 candidate sequence variation types. The sensitivity was 84.7%, the specificity was 83.3%, and positive predictive value was 98.3% (FIG. 13, part (c)), which indicates high discrimination performance and high performance as a system for discriminating between cancer and healthy subjects.

### Example 2

The following is a description of a method for discriminating cancer patients from healthy persons using sequence variation of nucleic acids in serum as an index, which can be carried out in a situation where there is no information on the sequence where sequence variation occurs in advance. The NGS analysis data described in Example 1 is used here. Without using previously obtained data, those in which sequence variation occurred in the actual NGS sequence information were used as the candidates for the sequence variation. The NGS sequence variation was detected as follows. The sequence of the miRBase Release 22 sequence was regarded as the wild type, that is, the sequence in which sequence variation had not occurred. Then, the miRNAs in the population aligned to the same miRNA, in which sequence variation occurred and the patterns of variation thereof were comprehensively detected and used. Alternatively, the miRNAs in which sequence variation occurred and their variation patterns were output in the VCF file format.

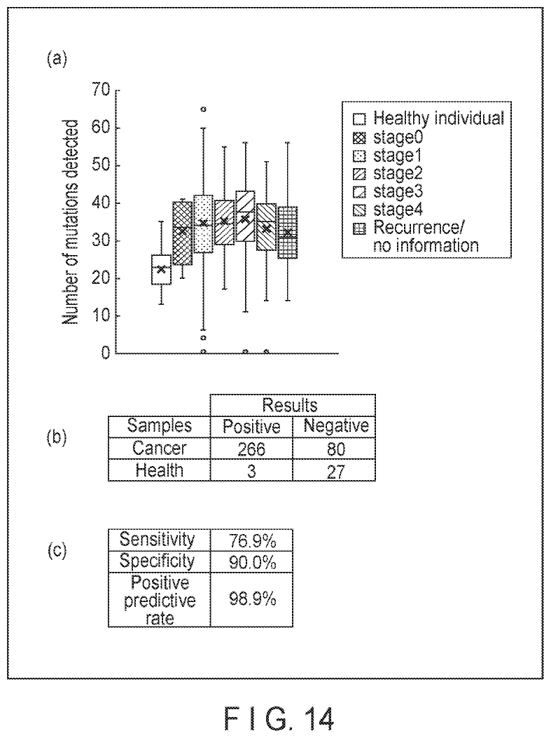

In this case, as in the case of Example 1, 10 bp at the 5′ end of miRNAs was focused and thus 310 types of sequence variation candidates could be selected. The results are shown in FIG. 14.

As in the case of Example 1, each sample was analyzed for the presence/absence of sequence variation. As a result, the AUC value was 0.854 (95% confidence interval 0.802-0.906). The threshold was set so that a subject was determined to have cancer if sequence variation was confirmed in 28 or more of the 310 candidate sequence variation types. Here, the sensitivity was 76.9%, specificity was 90.0%, and positive predictive value was 98.9% (FIG. 14, part (c)), which indicates high discrimination performance and high performance as a system discriminating between cancer and healthy subjects.

### Example 3

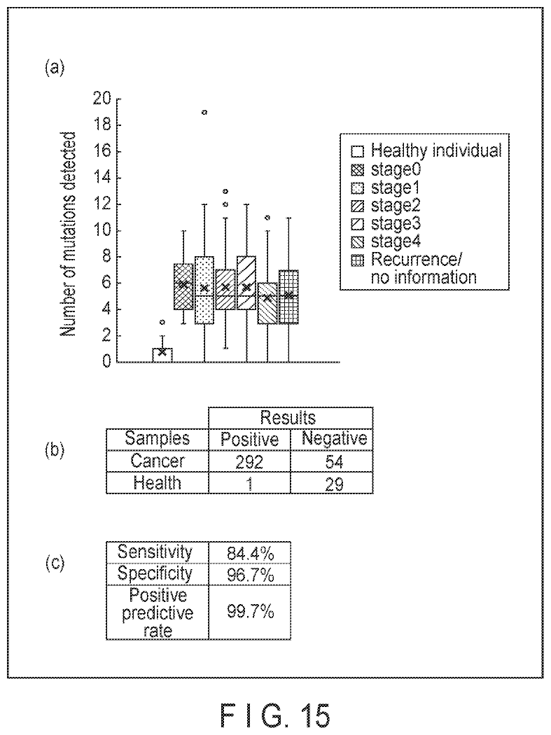

The following is a description of a method for discriminating cancer patients from healthy subjects using sequence variation of nucleic acids in serum as an index, using the selected markers. The NGS analysis data described in Example 1 is used here. When a similar analysis was carried out while limiting to the 24 types that specifically contributed to the discrimination between cancer and healthy subjects in Examples 1 and 2, the AUC value was 0.953 (95% confidence interval; 0.931-0.976). The results are shown in FIG. 11. The threshold was set so that a subject was determined to have cancer if sequence variation was confirmed in 3 or more of the 24 candidate sequence variation types. Here, the sensitivity was 84.4%, specificity was 96.7%, and positive predictive value was 99.7% (FIG. 15, part (c)), which indicates high discrimination performance and high performance as a system discriminating between cancer and healthy subjects, even in the case where the analysis was carried out with respect to the limited miRNAs.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

Further embodiments will be provided as additional notes.

(1) A method for analyzing the probability of suffering from cancer in a subject, comprising: counting the number of types of RNA in a sample derived from the subject with respect to the types of RNAs in which sequence variation caused by RNA editing exists in comparison with a reference sequence, and determining the probability of suffering from cancer in the subject by using the number of types of RNAs obtained as an index.

(2) The analytic method of item (1), wherein the sample derived from the subject is a body fluid, and the counting and the determining comprise:
acquiring sample data by acquiring information on sequence variation from a reference sequence from the body fluid;
extracting features by counting the number of types of RNAs in which the sequence variation exists; and
determining morbidity of cancer by using the number of types of RNAs having sequence variation as an index to identify a possibility of suffering from cancer.

(3) The analytic method of item (1) or (2), further comprising: comparing the number of types of the RNA from the subject and the number of types of the RNA obtained from a sample derived from a non-cancer individual with each other.

(4) The analytic method of item (3), wherein
when the number of the types of RNAs from the subject is greater than the number of the types of RNAs from the non-cancer individual, the subject is identified as likely to have cancer, and
when the number of the types of RNAs from the subject is less than or equal to the number of the types of RNAs from the non-cancer individual, the subject is identified as unlikely to have cancer.

(5) The analytic method of any one of items (2) to (4), wherein the body fluid is blood, serum or plasma.

(6) The analytic method of any one of items (1) to (5), wherein the RNA is miRNA, mRNA, tRNA, or piRNA.

(7) The analytic method of any one of items (1) to (5), wherein the RNA is miRNA.

(8) The analytic method of any one of items (1) to (7), wherein the sequence variation is caused by RNA editing.

(9) The analytic method of any one of items (1) to (7), wherein the sequence variation is caused by an RNA editing enzyme.

(10) The analytic method of any one of items (1) to (9), wherein the sequence variation is a base substitution.

(11) A method for analyzing the probability of suffering from cancer in a subject, comprising:
classifying RNAs contained in a sample derived from a subject according to homology of a sequence thereof to a reference sequence;
determining a group of representative sequences having identical sequences in an RNA population classified for each reference sequence;
comparing each sequence in the representative sequence group with the corresponding reference sequence to detect sequence variation in each; and
counting the number of types of representative sequences having the sequence variation.

(12) The analytic method of any one of items (1) to (11), wherein the cancer is at least one type of cancer selected from the group consisting of breast cancer, colorectal cancer, lung cancer, stomach cancer, pancreatic cancer, cervical cancer, uterine cancer, ovarian cancer, sarcoma, prostate cancer, bile duct cancer, bladder cancer, esophagus cancer, liver cancer, brain tumor, and kidney cancer.

(13) The method for analyzing the probability of suffering from cancer in a subject, as recited in any one of items (1) to (11), further comprising:
classifying RNAs contained in each of samples derived from a subject or a cancer individual and from a non-cancer individual, by homology of the RNA with respect to a reference sequence;
determining a representative sequence group having identical sequences in the RNA population classified for the reference sequence;
comparing each sequence of the representative sequence group to each corresponding reference sequence for samples derived from the subject or the cancer individual and samples derived from controls, and detecting the sequence variation;
counting the number of types of representative sequences having the sequence variation; and
comparing the number of types of the representative sequences counted in the subject or cancer individual and the number of types of the representative sequences counted in the controls, and determining a threshold for discriminating the subjects or the cancer individuals from the controls.

(14) The method for analyzing the probability of suffering from cancer in the subject as recited in any one of items (1) to (12), wherein the threshold value is greater than the number obtained for the sequence variation in the group of cancer patients than the number obtained from the group of non-cancer individuals.

(15) The method for analyzing the probability of suffering from cancer in the subject as recited in any one of items (1) to (12), wherein the threshold value is an integer greater than or equal to 2.

(16) The method for analyzing the probability of suffering from cancer in the subject as recited in any one of items (1) to (12), wherein the threshold value is determined by a method and the type of RNA used therein.

(17) A method for detecting sequence variation, comprising:
classifying RNAs contained in a sample derived from a subject by homology of the sequence thereof to a reference sequence;
determining a group of representative sequences having identical sequences in an RNA population classified for each of the reference sequences;
comparing each sequence of the representative sequence group with a corresponding reference sequence, and detecting sequence variations; and
counting the number of types of the sequence variations.

(18) The method of item (17), wherein in a specific sequence of the representative sequence group, occurrence of sequence variation in comparison with a corresponding reference sequence, can be identified together with sequence information of the representative sequence.

(19) The method of any one of items (1) to (18), wherein at least one method from the group consisting of NGS, qPCR, Sanger sequencing, microarray, and hybridization is selected and used.

What is claimed is:

1. A method for analyzing the probability of suffering from cancer in a subject, comprising:
counting the number of types of RNA in a sample derived from the subject with respect to the types of RNAs in which sequence variation based on RNA editing exists in comparison with a reference sequence; and
determining the probability of suffering from cancer in the subject by using the number of types of RNAs obtained as an index.

2. The analytic method of claim 1, wherein the sample derived from the subject is a body fluid, and the counting and the determining comprise:
acquiring sample data by acquiring information on sequence variation from a reference sequence from the body fluid;
extracting features by counting the number of types of RNAs in which the sequence variation exists; and
determining morbidity of cancer by using the number of types of RNAs having sequence variation as an index to identify a possibility of suffering from cancer.

3. The analytic method of claim 1, further comprising comparing the number of types of the RNA from the subject and the number of types of the RNA obtained from a sample derived from a non-cancerous individual with each other.

4. The method of claim 3, wherein when the number of the types of RNAs from the subject is greater than the number of the types of RNAs from the non-cancerous individual, the subject is identified as likely to have cancer, and when the number of the types of RNAs from the subject is less than or equal to the number of the types of RNAs from the non-cancer individual, the subject is identified as unlikely to have cancer.

5. The method of claim 2, wherein
the body fluid is blood, serum or plasma.

6. The method of claim 1, wherein the RNA is miRNA, mRNA, tRNA, or piRNA.

7. The method of claim 3, wherein the RNA is miRNA, mRNA, tRNA, or piRNA.

8. The method of claim 4, wherein
the RNA is miRNA, mRNA, tRNA, or piRNA.

9. The method of claim 1, wherein
the sequence variation is caused by an RNA editing enzyme.

10. The method of claim 1, wherein
the sequence variation is a base substitution.

11. A method for analyzing probability of suffering from cancer in a subject, comprising:
classifying RNAs contained in a sample derived from a subject according to homology of a sequence thereof to a reference sequence;
determining a group of representative sequences having identical sequences in an RNA population classified for each of the reference sequence;
comparing each sequence in the representative sequence group with the corresponding reference sequence to detect sequence variation in each; and
counting the number of types of the representative sequences having the sequence variation.

12. The method of claim 1, wherein the RNA is miRNA and the cancer is at least one type of cancer selected from the group consisting of breast cancer, colorectal cancer, lung cancer, stomach cancer, pancreatic cancer, cervical cancer, uterine cancer, ovarian cancer, sarcoma, prostate cancer, bile duct cancer, bladder cancer, esophagus cancer, liver cancer, brain tumor, and kidney cancer.

13. The method for analyzing the probability of suffering from cancer in a subject, of claim 1, further comprising:
classifying RNAs contained in each of samples derived from a subject or a cancer individual and from a non-cancer individual, by homology of the RNA with respect to a reference sequence;
determining a representative sequence group having identical sequences in the RNA population classified for the reference sequence;
comparing each sequence of the representative sequence group to each corresponding reference sequence for samples derived from the subject or cancer individual and samples derived from controls, and detecting the sequence variation;
counting the number of types of representative sequences having the sequence variation; and
comparing the number of types of representative sequences counted in the subject or cancer individual and the number of types of representative sequences counted in the controls, and determining a threshold for discriminating the subjects or cancer individuals from the controls.

14. The method for analyzing the probability of suffering from cancer in the subject of claim 1, wherein
the determining as the index is performed by: classifying RNAs contained in each of samples derived from a subject or a cancer individual and from a non-cancer individual, by homology of the RNA with respect to a reference sequence;
determining a representative sequence group having identical sequences in the RNA population classified for the reference sequence;
comparing each sequence of the representative sequence group to each corresponding reference sequence for samples derived from the subject or cancer individual and samples derived from controls, and detecting the sequence variation;
counting the number of types of representative sequences having the sequence variation; and
comparing the number of types of representative sequences counted in the subject or cancer individual and the number of types of representative sequences counted in the controls, and determining a threshold for discriminating the subjects or cancer individuals from the controls, and
the threshold value is greater than the number obtained for the sequence variation in the group of cancer individuals than the number obtained from the group of non-cancer individuals.

15. The method for analyzing the probability of suffering from cancer in the subject of claim 14, wherein
the threshold value is an integer greater than or equal to 2.

16. The method for analyzing the probability of suffering from cancer in the subject of claim 14, wherein
the threshold value is determined by a method and the type of RNA used therein.

17. The method for analyzing the probability of suffering from cancer in the subject of claim 14,
wherein at least one method from the group consisting of NGS, qPCR, Sanger sequencing, microarray, and hybridization is selected and used.

18. A method for detecting sequence variation, comprising:
classifying RNAs contained in a sample derived from a subject by homology of the sequence thereof to a reference sequence;
determining a group of representative sequences having identical sequences in an RNA population classified for each of the reference sequence;
comparing each sequence of the representative sequence group with the corresponding reference sequence, and detecting sequence variations; and
counting the number of types of the sequence variations.

19. The method for detecting sequence variation, of claim 18, wherein
in a specific sequence of the representative sequence group, occurrence of sequence variation in comparison with a corresponding reference sequence, can be identified together with sequence information of the specific representative sequence.

20. The method for detecting sequence variation, of claim 19, wherein
at least one method from the group consisting of NGS, qPCR, Sanger sequencing, microarray, and hybridization is selected and used.
